(19) United States
(12) Patent Application Publication
(10) Pub. No.: US 2015/0064129 A1
(43) Pub. Date: Mar. 5, 2015

(54) WOUND HEALING COMPOSITION

(71) Applicant: Taiwan Biotech Co., Ltd., Taoyuan City (TW)

(72) Inventors: LiChin Lin, TAOYUAN CITY (TW); YuKao Cheng, TAOYUAN CITY (TW); YuHua Chen, TAOYUAN CITY (TW); ShuFang Chiang, TAOYUAN CITY (TW)

(73) Assignee: TAIWAN BIOTECH CO., LTD., Taoyuan City (TW)

(21) Appl. No.: 14/018,120

(22) Filed: Sep. 4, 2013

(51) Int. Cl. A61K 31/79; A61K 31/728

(52) U.S. Cl. CPC A61K 31/79; A61K 31/728
USPC 424/78.06

(57) ABSTRACT

The invention discloses a composition comprising hyaluronic acid with a high molecular weight and povidone-iodine in low amount that has advantageous efficacy in enhancing wound healing and maintains high stability. Also disclosed is a method of using the composition of the invention in healing wounds.

### FIELD OF THE INVENTION

The invention relates to a wound healing composition and a method for wound healing. Particularly, the composition comprises hyaluronic acid and povidone iodine.

### BACKGROUND OF THE INVENTION

In general, skin wounds, superficial or deep tissue, afflict a great number of people each year. A multitude of different types of skin wounds exist and vary in cause, severity, healing time, location, etc. For example, while certain wounds heal rapidly, i.e., are acute, many have a relatively prolonged healing time, i.e., are chronic and may persist for weeks, months and even years, where in certain instances a wound may persist that never heals. Accordingly, symptoms or manifestations of wounds may vary and may range from, for example, mild abrasions to more severe wounds such as open-skin wounds, e.g., open sores, pustules and ulcerations, and the like, which may be debilitating. Wounds may arise from a number of different causes such as falling and scraping of the skin, burns, vascular insufficiency, e.g., caused by age or disease, etc. Regardless of the cause or particular symptoms, skin wounds need to be treated properly in order to promote effective and efficient healing.

Iodine has long been recognized as an antimicrobial agent with outstanding effectiveness against a wide range of micro-organisms including Gram positive and Gram negative bacteria, mycobacteria, fungi, protozoa and viruses. Tincture of iodine, which is a hydro-alcoholic solution of elemental iodine (I₂) and sodium iodide (NaI), is well recognized as a degerming antiseptic and has been in use for presurgical prepping of skin for over one hundred years. However, it is highly irritating, corrosive and toxic when in contact with a body cavity, mucus membranes or wounds. It also has other undesirable side effects that make it unsuitable for wound treatment. These include potential for occasional hypersensitivity reactions, skin staining and unpleasant odor. Major advances in utilizing the antimicrobial efficacy of iodine while minimizing its tissue toxicity and other undesirable side effects were made with the advent of iodophors. Iodophors are readily dissociable, loose complexes of tri-iodide or iodine with polymers or surfactants. Iodophors not only increase the solubility of iodine in aqueous media but also reduce its chemical potential and vapor pressure, thereby reducing its undesirable side effects. The iodophors serve as reservoirs of iodine and function by slowly releasing iodine at the site of application. A well known and very widely used iodophor is the polyvinylpyrrolidone-iodine complex, which is also known as PVP-iodine. Povidone-iodine is utilized in commercially available disinfectant products that are widely used in hospitals for prepping of skin prior to surgery and as surgical scrubs and hand washes for health care personnel hand washes.

Hyaluronan or hyaluronic acid is a linear mucopolysaccharide constituted by N-acetyl-D-glucosamine and D-glucuronic acid repeating units. It is a straight-chain polymer with a molecular weight which may vary between 50,000 and 13,000,000 Da, according to the source from which it was obtained and the methods of preparation which were used. Hyaluronic acid was first found in the vitreous body of a bovine's eye by Meyer and Palmer in 1934, and was then found in other tissues such as extracellular matrix (ECM) and synovial fluid of the joints. Hyaluronic acid is a viscoelastic fluid filled in the space between cells and collagenous fibers and coated on some epidermal tissues. Hyaluronic acid plays an important role in a biological organism, firstly as a mechanical support of the cells of many tissues, such as the skin, the tendons, the muscles and cartilage. Hyaluronic acid also performs a role in numerous physiological functions, such as adhesion, development, cell motility, cancer, angiogenesis, moistening of tissues, lubrication, and cellular migration. Due to the unique physical and biological properties of hyaluronic acid, it is employed in eye and joint surgery and is being evaluated in other medical procedures. Products of hyaluronic acid have also been developed for use in orthopedics, rheumatology, and dermatology.

U.S. Pat. No. 7,732,655 relates to a wound dressing comprising a therapeutic agent such as iodine, and a barrier layer comprises a substantially continuous film comprising the substrate material, such as hyaluronic acid. However, the barrier layer in this prior art reference is for initially separating the therapeutic agent from a wound fluid in use rather than providing efficacy with iodine in improving wound healing. US 2005/0181025 provides a composition for wound healing and prevention of adhesion to the wound comprising a physiologically acceptable salt of hyaluronic acid having a molecular weight from 200,000 Da to 2,500,000 Da, iodine and potassium iodine.

However, there is a need to develop a composition with higher stability and better efficacy in wound healing.

### SUMMARY OF THE INVENTION

The invention provides a composition comprising hyaluronic acid with a molecular weight no less than 100 KDa and povidone-iodine at an amount from about 0.005% (w/w) to 0.1% (w/w) based on the total weight of the composition.

The invention also provides a method for wound healing, comprising administering the composition of claim 1 to a target site of a subject.

In some embodiment, the hyaluronic acid or its derivative has a molecular weight of about the hyaluronic acid or its derivative used in the invention has a molecular weight of about 100 KDa to about 3,000 KDa, 100 KDa to about 2,500 KDa, 100 KDa to about 2,000 KDa, 500 KDa to about 3,000 KDa, 500 KDa to about 2,500 KDa, 500 KDa to about 2,000 KDa, 500 KDa to about 1,500 KDa, 800 KDa to about 3,000 KDa, 800 KDa to about 2,500 KDa, 800 KDa to about 2,000 KDa, or about 800 KDa to about 1,500 KDa.

In some embodiment, the amount of the hyaluronic acid or its derivative ranges from about 0.8% (w/w) to about 2.5% (w/w), about 0.8% (w/w) to about 2.0% (w/w), about 0.8% (w/w) to about 1.5% (w/w) or about 0.8% (w/w) to about 1.2% (w/w).

In some embodiment, the povidone-iodine is incorporated into the composition at an amount from about 0.005% (w/w) to about 0.5% (w/w), about 0.005% (w/w) to about 0.25% (w/w), about 0.005% (w/w) to about 0.2% (w/w), about 0.005% (w/w) to about 0.1% (w/w), about 0.01% (w/w) to about 0.5% (w/w), about 0.01% (w/w) to about 0.25% (w/w), about 0.01% (w/w) to about 0.2% (w/w), about 0.01% (w/w) to about 0.1% (w/w), about 0.075% (w/w) to about 0.1% (w/w), about 0.075% (w/w) to about 0.25% (w/w), about 0.075% (w/w) to about 0.1% (w/w), about 0.01% (w/w) to about 0.5% (w/w), about 0.01% (w/w) to about 0.25% (w/w), about 0.01% (w/w) to about 0.2% (w/w) or about 0.01% (w/w) to about 0.1% (w/w).

In some embodiments, the wound treated by the composition of the invention includes surgical wounds; bites; burns; acid and alkali burns; cold burns (frostbite), sun burn, minor cuts, major cuts, abrasions, lacerations, wounds caused by gunshot or knife injury; wounds caused by congenital disorders; wounds following dental surgery; periodontal disease; wounds following trauma; tumour associated wounds, which can be classified as malignant cutaneous ulcers related to the primary tumour or metastases; ulcers, leg ulcers; foot ulcers; pressure sores and corneal wounds.

### BRIEF DESCRIPTION OF THE DRAWINGS

FIGS. 1 (A) and (B) show the pH values of the compositions 10HA-KI₃ and 10HA-PI and the compositions 100HA-KI₃ and 100HA-PI, respectively.

FIGS. 2 (A) and (B) show the appearance of the compositions freshly prepared (A) and after storage (B).

FIGS. 3(A) and (B) shows the wound healing time of diabetic rats (FIG. 3(A)) and the healing status at 2, 4 and 6 weeks (FIG. 3(B); “HA(H)+KI₃”, “HA (L)+KI₃”, “HA (H)+PI” and “HA (L)+PI” refer to “100HA-KI3”, “10HA-KI3”, “100HA-PI” and “10HA-PI”, respectively).

FIG. 4 shows the wound healing areas at 2, 4 and 6 weeks of the rats treated with the compositions; “♦”: 2 weeks, “▪”: 4 weeks, “▴”: 6 weeks and “x”: 8 weeks.

### DETAILED DESCRIPTION OF THE INVENTION

The invention surprisingly found that a composition comprising high molecular weight hyaluronic acid and povidone-iodine in a low amount has advantageous efficacy in enhancing wound healing. Particularly, the composition maintains high stability.

While the present invention is capable of being embodied in various forms, the description of several embodiments below is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

The term “povidone” is an art recognized synonym for polyvinylpyrrolidone (commonly used name as polymeric 1-vinyl-2-pyrrolidone), so it will be understood that the term “Povidone-iodine” is synonymous with, and an alternative way of referring to, a polyvinylpyrrolidone-iodine complex.

The term “hyaluronic acid” used herein refers to an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein. Also, the term “hyaluronic acid” as used herein includes alkali metal salts such as sodium, potassium and lithium salts of hyaluronic acid.

The term “aqueous medium” used herein means water, physiologic saline, a buffer solution or an aqueous solution containing an organic solvent such as an alcohol.

The term “pharmaceutically acceptable” as used herein means that the substance in question does not produce unacceptable toxicity to the subject or interaction with other components of the composition.

The term “administering” includes routes of administration which allow the composition of the invention to perform their intended function.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

The term “wound” used herein, refers to a disruption of the normal continuity of structures caused by a physical (e.g., mechanical) force, a biological (e.g., thermic or actinic force, or a chemical means. In particular, the term “wound” encompasses wounds of the skin.

The term “wound” also encompasses contused wounds, as well as incised, stab, lacerated, open, penetrating, puncture, abrasions, grazes, burns, frostbites, corrosions, wounds caused by ripping, scratching, pressure, and biting, and other types of wounds. In particular, the term encompasses ulcerations (i.e., ulcers), preferably ulcers of the skin.

The term “wound healing” as used herein refers to a regenerative process with the induction of an exact temporal and spatial healing program comprising wound closure and the processes involved in wound closure. The term “wound healing” encompasses but is not limited to the processes of granulation, neovascularization, fibroblast, endothelial and epithelial cell migration, extracellular matrix deposition, re-epithelialization, and remodeling.

In one aspect, the invention provides a composition comprising hyaluronic acid with a molecular weight no less than 100 KDa and povidone-iodine at an amount from about 0.005% (w/w) to 0.1% (w/w) based on the total weight of the composition.

### Hyaluronic Acid or Derivatives Thereof

Hyaluronic acid or its derivative with a molecular weight no less than 100 KDa can be used in the hydrogel composition of the invention. In one embodiment, the hyaluronic acid or its derivative used in the invention has a molecular weight of about 100 KDa to about 3,000 KDa, 100 KDa to about 2,500 KDa, 100 KDa to about 2,000 KDa, 500 KDa to about 3,000 KDa, 500 KDa to about 2,500 KDa, 500 KDa to about 2,000 KDa, 500 KDa to about 1,500 KDa, 800 KDa to about 3,000 KDa, 800 KDa to about 2,500 KDa, 800 KDa to about 2,000 KDa, or about 800 KDa to about 1,500 KDa. In a preferred embodiment, the hyaluronic acid or its derivative used in the invention has a molecular weight of about 800 KDa to about 3,000 KDa, 800 KDa to about 2,500 KDa, 800 KDa to about 2,000 KDa, or about 800 KDa to about 1,500 KDa. In a more preferred embodiment, the hyaluronic acid used in the invention has a molecular weight of about 1,000 KDa to about 1,500 KDa, 1,000 KDa to about 2,000 KDa or 1,000 KDa to about 3,000 KDa. In a more preferred embodiment, the hyaluronic acid used in the invention has a molecular weight of about 1,000 KDa, about 1,500 KDa, about 2,000 KDa, about 2,500 KDa or about 3,000 KDa. The average molecular weight of the hyaluronic acid may be determined using standard methods in the art, such as those described by Ueno et al, 1988, Chem. Pharm. Bull. 36, 4971-4975; Wyatt, 1993, Anal. Chim. Acta 272:1-40; and Wyatt Technologies, 1999, “Light Scattering University DAWN Course Manual” and “DAWN EOS Manual” Wyatt Technology Corporation, Santa Barbara, Calif.

The “hyaluronan” or “hyaluronic acid” is acidic polysaccharides with different molecular weights constituted by residues of D-glucuronic and N-acetyl-D-glucosamine acids. Any source of the hyaluronic acid or its derivative can be used in the invention. Rooster combs are a significant commercial source for hyaluronan. Microorganisms are an alternative source. U.S. Pat. No. 4,801,539 and European Patent No. EP0694616 disclose a fermentation method for preparing hyaluronic acid involving a strain of Streptococcus zooepidemicus. As disclosed in WO 03/054163 (Novozymes), hyaluronic acid or salts thereof may be recombinantly produced, e.g., in a Gram-positive Bacillus host.

The hyaluronic acid derivatives include, but are not limited to (1) the esters of hyaluronic acid, wherein part or all of the carboxy functions are esterified with alcohols of the aliphatic, aromatic, arylaliphatic, cycloaliphatic, heterocyclic series (EP 0 216 453 B1); (2) the autocross-linked esters of hyaluronic acid, wherein part or all of the carboxy groups are esterified with the alcoholic functions of the same polysaccharide chain or of other chains (EP 0 341 745 B1); (3) the cross-linked compounds of hyaluronic acid, wherein part of all of the carboxy groups are esterified with polyalcohols of the aliphatic, aromatic, arylaliphatic, cycloaliphatic, heterocyclic series, generating cross-linking by means of spacer chains (EP 0 265 116 B1); (4) the hemiesters of succinic acid or the heavy metal salts of the hemiester of succinic acid with hyaluronic acid or with partial or total esters of hyaluronic acid (WO 96/357201); (5) the O-sulphated derivatives (WO 95/25751) or O/N-sulphated derivatives (WO 98/45335); and (6) the amidic derivatives of hyaluronic acid or of the above-listed compounds.

The hyaluronic acid derivatives can be total or partial esters with alcohols of the aliphatic, aromatic, arylaliphatic, cycloaliphatic or heterocyclic esters. Alcohols of the aliphatic series to be used as esterifying components of the carboxylic groups of hyaluronic acid are, for example, those with a maximum of 34 carbon atoms, which may be saturated or unsaturated and which may possibly also be substituted by other free functional or functionally modified groups, such as amine, hydroxyl, aldehyde, ketone, mercaptan, or carboxyl groups or by groups derived from these, such as hydrocarbyl or di-hydrocarbylamine groups (from now on the term “hydrocarbyl” will be used to refer not only to monovalent radicals of hydrocarbons, but also bivalent or trivalent radicals, ether or ester groups, acetal or ketal groups, thioether or thioester groups, and esterified carboxyl or carbamide groups and carbamide substituted by one or more hydrocarbyl groups, by nitrile groups or by halogens.

In one embodiment, the amount of the hyaluronic acid or its derivative used in the invention ranges from about 0.8% (w/w) to about 2.5% (w/w), about 0.8% (w/w) to about 2.0% (w/w), about 0.8% (w/w) to about 1.5% (w/w) or about 0.8% (w/w) to about 1.2% (w/w). Preferably, the amount ranges from about 1.0% (w/w) to about 2.0% (w/w), 1.0% (w/w) to about 1.8% (w/w) or 1.0% (w/w) to about 1.7% (w/w), 1.0% (w/w) to about 1.6% (w/w), 1.0% (w/w) to about 1.5% (w/w), 1.0% (w/w) to about 1.4% (w/w), 1.0% (w/w) to about 1.3% (w/w) or 1.0% (w/w) to about 1.2% (w/w). More preferably, the amount is about 1.0% (w/w), about 1.1% (w/w), about 1.2% (w/w), about 1.3% (w/w), about 1.4% (w/w) or about 1.5% (w/w).

The level of hyaluronic acid may be determined according to the modified carbazole method (Bitter and Muir, 1962, Anal Biochem. 4:330-334).

### Povidone Iodine

Povidone-iodine used in the composition of the present invention may bevarious commercially available products, for example, PVP-IODINE 30/06 (manufactured by BASF), PVP-Iodine 10 (manufactured by GAF), etc.

The povidone-iodine is incorporated into the composition at an amount from about 0.005% (w/w) to about 0.5% (w/w), about 0.005% (w/w) to about 0.25% (w/w), about 0.005% (w/w) to about 0.2% (w/w), about 0.005% (w/w) to about 0.1% (w/w), about 0.01% (w/w) to about 0.5% (w/w), about 0.01% (w/w) to about 0.25% (w/w), about 0.01% (w/w) to about 0.2% (w/w), about 0.01% (w/w) to about 0.1% (w/w), about 0.075% (w/w) to about 0.1% (w/w), about 0.075% (w/w) to about 0.25% (w/w), about 0.075% (w/w) to about 0.1% (w/w), about 0.01% (w/w) to about 0.5% (w/w), about 0.01% (w/w) to about 0.25% (w/w), about 0.01% (w/w) to about 0.2% (w/w) or about 0.01% (w/w) to about 0.1% (w/w), preferably from about 0.01% (w/w) to about 0.25% (w/w), more preferably about 0.01% (w/w) to about 0.1% (w/w); most preferably about 0.01% (w/w), based on the total weight of the composition. The inventors surprisingly found that the providone-iodine at the above-mentioned amounts in combination with the hyaluronic acid with the molecular weights herein maintains advantageous stability and efficacy in wound healing. If the amount of providone-iodine is less than 0.005% by weight, the microbicidal effect would be insufficient and the stability of the povidone-iodine could not be maintained. If the amount is more than 0.5% by weight, the offensive or would become unpleasantly too strong and, in addition, when the composition is applied to a substance to be protected from microbes, the substance would be inconveniently colored or the appearance of the color of the substance itself would often be strengthened too much because of the application of the composition thereto so that the commercial value of the substance would be reduced.

### Hydrogel Composition of the Invention and its Applications

The compositions of the invention are designed to offer a combination of high molecular weight hyaluronic acid and povidone-iodine in a low amount that has advantageous efficacy in enhancing wound healing. The composition also maintains high stability. The preferred embodiments of the high molecular weight hyaluronic acid and povidone-iodine in a low amount are as mentioned above.

The term “wound” and “tissue injury” can be used interchangeably. The wounds or tissue injury may include, but are not limited to the following: surgical wounds; bites; burns; acid and alkali burns; cold burn (frostbite), sun burn, minor cuts, major cuts, abrasions, lacerations, wounds caused by gunshot or knife injury; wounds caused by congenital disorders; wounds following dental surgery; periodontal disease; wounds following trauma; tumour associated wounds, which can be classified as malignant cutaneous ulcers related to the primary tumour or metastases; ulcers, leg ulcers; foot ulcers; pressure sores and corneal wounds. For example, the composition of the invention may be used to treat an injury which causes epidermal damage such as incisions, wounds in which the skin is broken by a cutting instrument, and lacerations, wounds by which the skin is broken by a blunt or dull instrument, and wounds of the skin caused by friction. The composition of the invention may also be used to treat dermatological disorders such as burns, Candidiasis and diaper rash, donor and receptor site wounds for skin transplants, ulcers (cutaneous, decubitus, venous stasis, sickle cell, and diabetic), psoriasis, skin rashes, and sunburn photo-reactive processes, and second and third degree burns. The composition of the invention may also be used to protect or accelerate the healing of oral tissue such as mouth sores, burns, surgical sites, and ulcerations. The composition of the invention may also be used to treat wounds such as those which result from corneal ulcers, radial keratotomy, corneal transplants, epikaratophakia and other surgically induced wounds in the eye. In a preferred embodiment, the composition of the invention is used to treat wounds such as contusion, incisions and lacerations.

Accordingly, the invention provides a method for wound healing, comprising administering the composition of the invention to a target site of a subject.

Wound healing can be achieved by administering the compositions of the invention externally. Preferably, the compositions of the present invention may further include a pharmaceutically acceptable carrier. For example, the composition of the present invention could be externally administered with an acceptable carrier in the form of a gel, lotion, cream, tonic, emulsion, etc. For example, an acceptable carrier may act variously as a solvent, carrier, diluent or dispersant for the constituents of the composition, which allows for the uniform application of the ingredients to the target at an appropriate dilution. The acceptable carrier may also facilitate penetration of the composition into the skin. Preferably the acceptable carrier used in practicing the present invention comprises water and ethanol.

The compositions of the invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The dose of the composition of the invention applied to the target will depend upon the particular wound or tissue injury suffered, the age and overall condition of the subject, the route of administration, etc., and can be optimized in accordance with known techniques.

In addition to the wound healing, the composition of the invention maintains high stability. Wound healing is a complex physiological process which is impaired in the chronic wound. A factor which influences wound healing is the pH environment. Both acute and chronic wounds move to a neutral and then acidic state as healing occurs. Generally, an acidic environment is not beneficial to bacterial growth and can inhibit activity of matrix-metalloproteases, so it is beneficial to wound healing. On the contrary, a basic environment is beneficial to bacterial growth and can cause matrix-metalloproteas to become over active, so it makes wound healing difficult. The composition of the invention has low pH variation and maintains high stability.

### EXAMPLES

### Example 1

### Preparation of the Composition of the Invention

The hyaluronic acid (HA) stock solution is prepared by respectively solving 1.5 g of hyaluronic acid with 100 KDa, 1,000 KDa and 2,000 KDa in 80 g of deionized water (DI water) and then filtered for sterilization. The povidone-iodide (PI) stock solution was prepared by adding 0.05 g of povidone-iodide in DI water to a total weight of 100 g and then filtered for sterilization. The KI₃ stock solution was prepared by adding 0.75 g of KI₃ and 0.5 g of iodine in DI water to a total weight of 100 g and then filtered for sterilization (positive control group). The following samples were prepared by diluting the above stock solutions and then mixing the resultant solutions.

| | HA-KI | HA-PI (0.01) | HA-PI (0.1) | HA-PI (0.25) |
|---|---|---|---|---|
| Sodium | 1.5 g | 1.5 g | 1.5 g | 1.5 g |
| Hyaluronate | | | | |
| (100 Kda or | | | | |
| 1,000 KDa) | | | | |
| Potassium | 0.15 g | — | — | — |
| iodide (KI) | | | | |
| Iodine (I₂) | 0.1 g | — | — | — |
| Povidone-iodine | — | 0.01 g | 0.1 g | 0.25 g |
| (PI) | | | | |
| Injection water | Ad 100 g | Ad 100 g | Ad 100 g | Ad 100 g |

### Example 2

### pH Stability of the Composition of the Invention

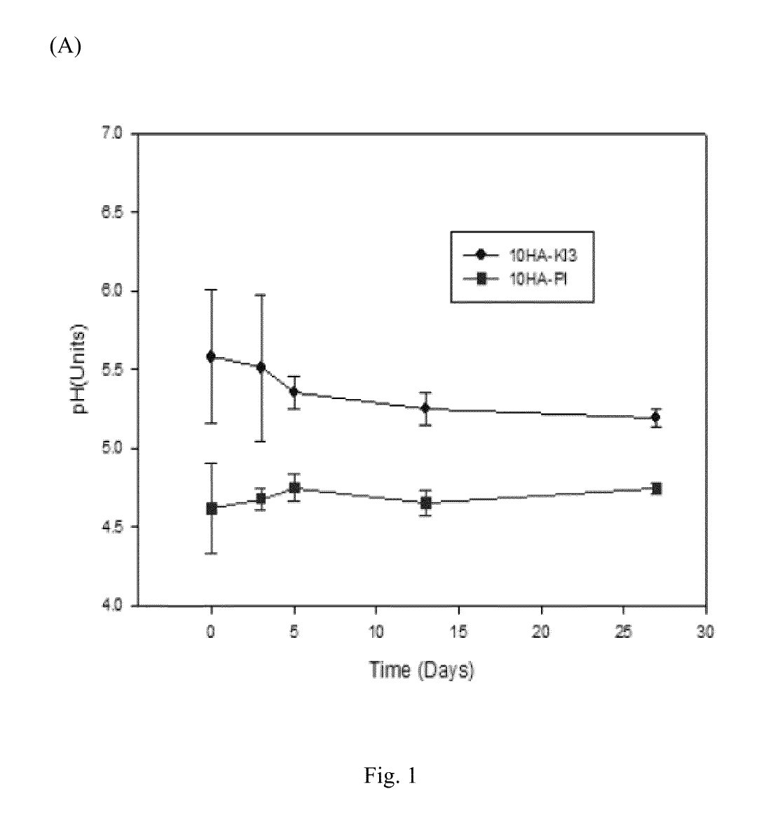

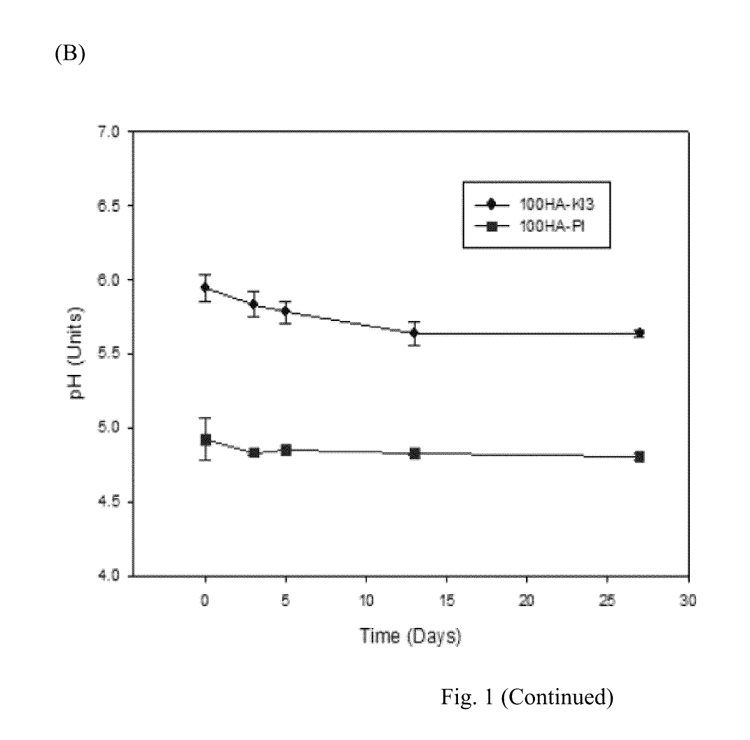

pH values of the following compositions 10HA-KI, 100HA-KI, 10HA-PI and 100HA-PI were detected at different time points (0 day to 27 days) by pH meter (EUTECH PH510) and the results are shown in FIG. 1 (A) for the compositions 10HA-KI₃ and 10HA-PI and FIG. 1 (B) for 100HA-KI₃ and 100HA-PI. The pH values at 0 day and 27 day of the above-mentioned composition are shown in Table 1.

TABLE 1

| | Sample | | | |
|---|---|---|---|---|
| | 10HA-KI | 100HA-KI | 10HA-PI | 100HA-PI |
| HA molecular | 100 kDa | 1,000 kDa | 100 kDa | 1,000 kDa |
| weight | | | | |
| Iodine source | KI₃ | KI₃ | Povidone-iodine | Povidone-iodine |
| | complex | complex | | |
| Initial pH value | 5.6 | 6.0 | 4.6 | 4.9 |
| at 0 day | | | | |
| Final pH value | 4.9 | 5.5 | 4.5 | 4.7 |
| at 27 day | | | | |

As shown in the table, the pH variation of the composition (10HA-KI) comprising low molecule weight HA (100 KDa) and KI₃ is about 17.8%, that of the composition (100HA-KI) comprising high molecule weight HA (1,000 KDa) and KI₃ is about 7.4% and that of the composition (10HA-PI) comprising low molecule weight HA (100 KDa) and providone iodine is about 6.6%, while there is no pH variation for the composition (100HA-PI) comprising high molecule weight HA (1,000 KDa) and providone iodine. As a result, the compositions comprising HA and PI have significantly higher stability than the compositions comprising HA and KI₃ complex wherein the stability of 100HA-PI is slightly higher than that of 10HA-PI. The composition (200HA-KI) comprising HA with 2,000 KDa molecular weight and KI₃ and the composition (200HA-PI) comprising HA with 2,000 KDa molecular weight and povidone-iodine was prepared according to the process mentioned in Example 1. After 30 days, the pH variation of 200HA-KI is about 3%, whereas that of 200HA-PI is about 0.8%. Given the above, the composition comprising PI and higher molecular weight HA is also more stable than that comprising KI₃ complex and higher molecular weight HA.

### Example 3

### Appearance of the Composition of the Invention

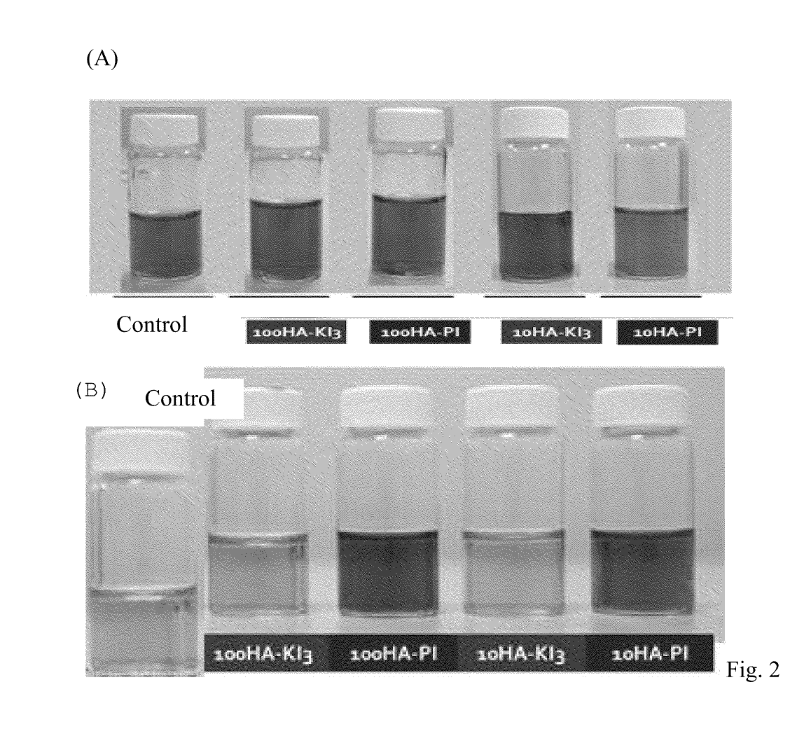

The appearance of the freshly prepared samples in Example 2 exhibits reddish brown. After the samples in Table 1 were stored away from light and at room temperature for 5 months, the compositions containing HA and KI₃ exhibit yellow brown, whereas the compositions containing HA and povidone-iodine still exhibit reddish brown but the sample of HA with high molecular weight is darker in color (see FIGS. 2 (A) and (B)). The appearance analysis also shows that the compositions comprising high molecular weight HA and PI have significantly high stability than that comprising HA and KI₃ complex.

The formulation in the example of US 2005/0181025A1 was further used as comparative experiment. The formulation is as follows:

| Ingredient | weight |
|---|---|
| Sodium hyaluronan | 1.5 g (1.5%) |
| KI | 0.15 g (0.15%) |
| I₂ | 0.1 g (0.1%) |
| Injection water | ad. 100 g |

After it was stored away from light and at room temperature for 5 months, the appearance exhibited a yellow-brown.

### Example 4

### Wound Healing Assay in Animal Model

Forty-four Wister male rats with diabetes were used for the experiments and fed with a standard balanced pellet diet with water ad libitum. Rats were marked to permit individual identification and were kept in their cages for 14 days prior to experimentation to allow for acclimatization to the laboratory conditions.

The rats were anesthetized by intraperitoneal injection with 6% chloral hydrate (5 mL/kg, Riedel-deHaën®, Schnelldorf, Germany). A full-thickness wound of 6×5 cm² was generated on the back of the rat with a sterile disposable biopsy punch. The rats were divided intoseven groups and the wounds were treated with a phosphate buffer dressing as a control, HA dressing, HA-KI dressing and HA-povidone-iodine (0.01%) dressing as shown in Table 2.

TABLE 2

| | 100HA | 10HA |
|---|---|---|
| Control (PBS) | 8 rats | |
| HA alone | 6 rats | 6 rats |
| HA-iodine complex | 6 rats | 6 rats |
| HA-povidone-iodine (0.01%) | 6 rats | 6 rats |

The time needed for wound healing of the diabetic rats is shown in Table 3.

TABLE 3
Wound healing time (wks)

| Condition | Control | 100HA-KI₃ | 10HA-KI₃ | 100HA-PI | 10HA-PI |
|---|---|---|---|---|---|
| Average | 9.8 | 8.3 | 9.0 | 6.3 | 8.3 |
| SD | 0.8 | 1.8 | 1.4 | 1.0 | 1.0 |
| Numbers | 6 | 5 | 5 | 6 | 6 |

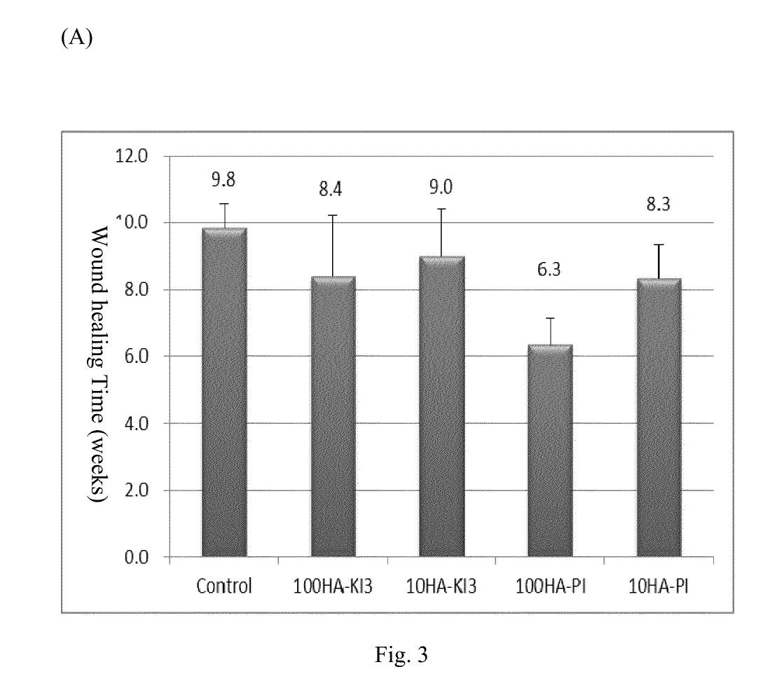

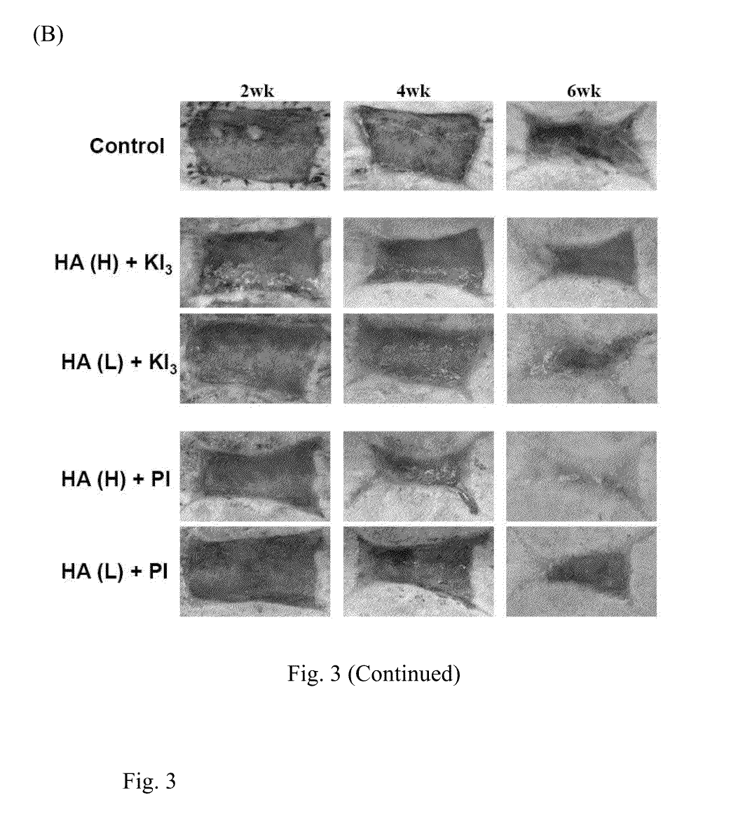

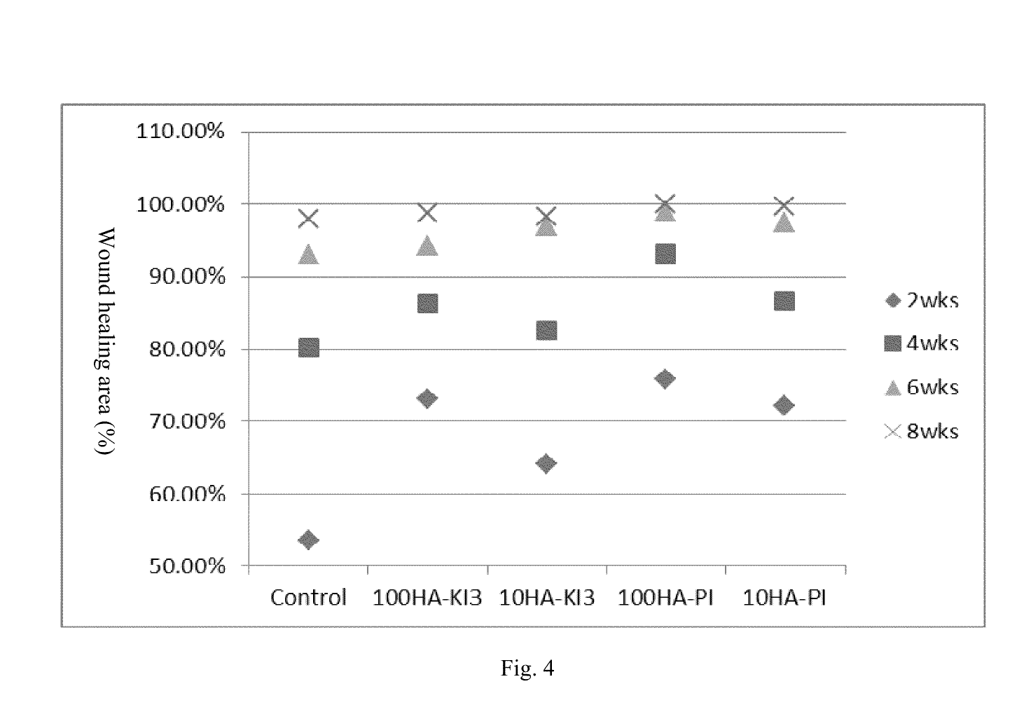

FIGS. 3 (A) and (B) show the wound healing time of the diabetic rats and the photos of the healing status at 2, 4 and 6 weeks, respectively. In this assay, the time needed for wound healing in the control group of diabetic rats was about 9.8+/−0.8 weeks on average, whereas the 10HA-PI and 100HA-PI shortened the time needed for wound healing to 8.3+/−1 weeks and 6.3+/−1 weeks, respectively. In addition, the times needed for wound healing in the HA-PI groups are also shortened than those in the HA-KI₃ groups. The wound healing areas at 2, 4 and 6 weeks of the rats treated with the samples in Table 2 are shown in Table 4 and FIG. 4.

Wound healing area (%)

| | 2 weeks | | 4 weeks | | 6 weeks | | 8 weeks | |
|---|---|---|---|---|---|---|---|---|
| Control | 53.6% | — | 80.1% | — | 93.0% | — | 98.0% | — |
| 100HA- | 73.1% | +19.5% | 86.2% | +6.1% | 94.3% | +1.3% | 98.7% | +0.7% |
| KI₃ | | | | | | | | |
| 10HA-KI₃ | 64.2% | +10.6% | 82.5% | +2.4% | 97.0% | +4.0% | 98.2% | +0.2% |
| 100HA-PI | 75.9% | +22.3% | 93.1% | +13.0% | 98.9% | +5.9% | 100% | +2.0% |
| 10HA-PI | 72.2% | +18.6% | 86.6% | +6.5% | 97.5% | +4.5% | 99.7% | +1.7% |

As shown in the table and figure, after 4 weeks of surgery, the wound healing area of 100HA-PI is 93.1% and that of the PBS is only 80.1%. After 6 weeks, the wound healing area of 100HA-PI is the largest and reaches about 100% after 8 weeks.

What is claimed is:

1. A wound-healing composition comprising hyaluronic acid with a molecular weight no less than 100 KDa and povidone-iodine at an amount from about 0.005% (w/w) to 0.1% (w/w) based on the total weight of the composition.

2. The composition of claim 1, wherein the hyaluronic acid or its derivative has a molecular weight of about 100 KDa to about 3,000 KDa.

3. The composition of claim 1, wherein the hyaluronic acid or its derivative has a molecular weight of about 1,000 KDa to about 3,000 KDa.

4. The composition of claim 1, wherein the hyaluronic acid or its derivative has a molecular weight of about 1,000 KDa or about 2,000 KDa.

5. The composition of claim 1, wherein the amount of the hyaluronic acid or its derivative ranges from about 0.8% (w/w) to about 2.5% (w/w).

6. The composition of claim 1, wherein the amount of the hyaluronic acid or its derivative ranges from about 1.0% (w/w) to about 2.0% (w/w).

7. The composition of claim 1, wherein the amount of the hyaluronic acid or its derivative is about 1.5% (w/w).

8. The composition of claim 1, wherein the amount of povidone-iodine is about 0.01% (w/w) to about 0.25% (w/w).

9. The composition of claim 1, wherein the amount of povidone-iodine is about 0.01% (w/w) to about 0.1% (w/w).

10. The composition of claim 1, wherein the amount of povidone-iodine is about 0.01% (w/w).

11. The composition of claim 1, which is in the form of a gel, lotion, cream, tonic or emulsion.

12. The composition of claim 1, wherein the wound includes surgical wounds; bites; burns; acid and alkali burns; cold burns (frostbite), sun burn, minor cuts, major cuts, abrasions, lacerations, wounds caused by gunshot or knife injury; wounds caused by congenital disorders; wounds following dental surgery; periodontal disease; wounds following trauma; tumour associated wounds, which can be classified as malignant cutaneous ulcers related to the primary tumour or metastases; ulcers, leg ulcers; foot ulcers; pressure sores and corneal wounds.

13. The composition of claim 1, wherein the wound includes that caused by dermatological disorders such as burns, Candidiasis and diaper rash, donor and receptor site wounds for skin transplants, ulcers (cutaneous, decubitus, venous stasis, sickle cell, and diabetic), psoriasis, skin rashes, and sunburn photo reactive processes, and second and third degree burns.

14. The composition of claim 1, wherein the wound includes corneal ulcers, radial keratotomy, corneal transplants, epikaratophakia and other surgically induced wounds in the eye.

15. A method for wound healing, comprising administering the composition of claim 1 to a target site of a subject.

16. The method of claim 15, wherein the wound includes surgical wounds; bites; burns; acid and alkali burns; cold burns (frostbite), sun burn, minor cuts, major cuts, abrasions, lacerations, wounds caused by gunshot or knife injury; wounds caused by congenital disorders; wounds following dental surgery; periodontal disease; wounds following trauma; tumour associated wounds, which can be classified as malignant cutaneous ulcers related to the primary tumour or metastases; ulcers, leg ulcers; foot ulcers; pressure sores and corneal wounds.

17. The method of claim 15, wherein the wound includes that caused by dermatological disorders such as burns, Candidiasis and diaper rash, donor and receptor site wounds for skin transplants, ulcers (cutaneous, decubitus, venous stasis, sickle cell, and diabetic), psoriasis, skin rashes, and sunburn photo reactive processes, and second and third degree burns.

18. The method of claim 15, wherein the wound includes corneal ulcers, radial keratotomy, corneal transplants, epikaratophakia and other surgically induced wounds in the eye.
